(19) United States
(12) Patent Application Publication
(10) Pub. No.: US 2026/0325198 A1
(43) Pub. Date: Oct. 22, 2026

(54) ELECTROSURGICAL DEVICE, SYSTEM, AND METHOD FOR CONTROLLING AN ELECTROSURGICAL DEVICE

(71) Applicant: KARL STORZ SE & Co. KG, Tuttlingen (DE)

(72) Inventors: Jérôme Pierre, Tuttlingen (DE); Thomas HINDING, Tuttlingen (DE)

(21) Appl. No.: 19/122,018

(22) PCT Filed: Oct. 19, 2023
(86) PCT No.: PCT/EP2023/079093

§ 371 Date: Apr. 17, 2025

(30) Foreign Application Priority Data
Oct. 21, 2022 (DE) 10 2022 127 935.9

(51) Int. Cl. A61B 18/12; A61B 18/00

(52) U.S. Cl. CPC A61B 18/1233; A61B 2018/00589; A61B 2018/00601; A61B 2018/00607; A61B 2018/00761; A61B 2018/00767; A61B 2018/00892

(57) ABSTRACT

An electrosurgical device (1) for high-frequency generation, includes a high-frequency signal generator (3), which has a plurality of output stages (5), each end output stage (5) including an electronic switch (7). Each of the electronic switches (7) is configured to be switched depending on at least one target parameter. The high-frequency signal generator (3) is configured to generate, by pulse duration modulation, from the totality of the voltage curves generated by the electronic switches (7), a step-shaped voltage curve including pulse-width-modulated square wave voltages, which curve follows a sine reference signal. A control unit (4) is configured to regulate an output voltage for actuating the output stages (5) depending on the pulse-width-modulated square wave voltages.

### CROSS REFERENCE TO RELATED APPLICATIONS

This application is a United States National Phase Application of International Application PCT/EP2023/079093, filed Oct. 19, 2023, and claims the benefit of priority under 35 U.S.C. § 119 of German Application 10 2022 127 935.9, filed Oct. 21, 2022, the entire contents of which are incorporated herein by reference.

### TECHNICAL FIELD

The invention relates to an electrosurgical device for high-frequency generation. Furthermore, the invention relates to a system comprising an electrosurgical device and an electrosurgical instrument. The invention further relates to a method for controlling an electrosurgical device.

### BACKGROUND

In high-frequency surgery (HF surgery) or electrosurgery, a high-frequency alternating current is passed through the human body in order to specifically damage or cut tissue by the heating causing thereby.

In minimally invasive endoscopy, electrosurgical coagulation or cutting instruments are used. This requires fast and reliable tissue sealing. Even a small amount of blood is enough to obstruct a surgeon's view and make it difficult or even impossible to operate on the patient. In principle, tissue sealing involves heating tissue using a high-frequency current. As a result of being heated, the tissue is sealed. This makes it possible to immediately stop any bleeding in the tissue that may occur.

The current used for heating must have a certain frequency to prevent nerve irritation. In other words, the nerves are not intended to be stimulated. Typically, appropriate frequencies are in the kHz range.

A transformer can be used to achieve the required voltage. The transformer is typically operated by means of a half or full bridge on a primary side with a frequency of at least 150 kHz. A voltage can be applied on a secondary side and can be used, for example, to coagulate or cut tissue. In this case, the tissue to be treated must not be heated too strongly or for too long, as this may lead to carbonization or the formation of eschars, which can trigger further uncontrollable bleeding. However, accurate or reliable voltage control is difficult.

In the case of bi-polar systems known from the prior art, it is necessary that radio detection must also be present.

The frequency described above is generated by means of a half or full bridge. However, due to the high power, the components used can only be operated digitally, so they can only be switched on or off. A transformer can ideally be operated with a sine wave. “Hard” interconnection of the transistors used can cause many harmonics, i.e., disturbances that can become critical in an EMC test on the one hand and can also make exact measurements of current-voltage phase shifts considerably more difficult and inaccurate on the other hand.

At lower frequencies, a delta modulation method can be used. However, this is not possible at higher frequencies because the transistors cannot switch quickly enough.

For example, a high-frequency surgical generator is known from WO 2010/025807 A1.

### SUMMARY

The object of the present invention is to overcome the disadvantages of the prior art.

Preferably, it should be possible, by means of the invention, to generate a clean signal which has only low power losses in the electronics, and which generates no or few harmonics and which can be controlled quickly, safely, precisely and reliably.

This object is achieved by an electrosurgical device for high-frequency generation with a frequency of at least 150 kHz for coagulation and/or cutting of tissue, comprising
a primary circuit with a high-frequency signal generator, the high-frequency signal generator having a plurality of output stages,
a secondary circuit, and
a transformer, the primary circuit being galvanically isolated from the secondary circuit by means of the transformer,
each output stage comprising an electronic switch, each of the electronic switches being configured to be switched depending on at least one target parameter,
the high-frequency signal generator being configured to generate a step-shaped voltage curve from pulse-width-modulated square wave voltages from the totality of the voltage curves generated by the electronic switches, by means of pulse duration modulation, the step-shaped voltage curve following a sine reference signal, and
a control unit configured to regulate an output voltage for controlling the output stages depending on the pulse-width-modulated square wave voltages.

As a result of the use of a plurality of electronic switches that can be interconnected one after the other, a high frequency can be generated. This makes it possible to generate a sine wave with a frequency of at least 150 kHz, preferably at least 200 kHz, preferably at least 300 kHz.

Preferably, the electronic switch is selected from the group comprising an electrical switch, an electrical circuit, a transistor or a MOS-FET.

The electrosurgical device can generate a clean signal that has only minimal power losses in the electronics, produces few or no harmonics, and can be controlled quickly, safely, and reliably.

A primary circuit can be understood as one device side of the device. A secondary circuit can be understood as the side facing away from the primary circuit, to which an instrument can be connected. An instrument, e.g., coagulation forceps, can be arranged or connected in the secondary circuit.

Galvanic isolation (also called galvanic decoupling or electrical isolation) is understood to mean avoiding electrical conduction between two circuits between which power or signals are to be exchanged. In the case of galvanic isolation, electrical potentials are separated from each other and the circuits are then potential-free with respect to each other. This isolation may not be stopped at any other point, for example via grounding. The electrical conduction is preferably separated by electrically non-conductive coupling elements.

A target parameter can be understood as an “and” connection. Preferably, two conditions must be met to enable switching.

Switching can be understood as interconnection or energizing.

The control unit can be a central control circuit, a controller or a control device. The control unit can also be a closed-loop control unit, a closed-loop control circuit or a closed-loop control device. “At least” can be understood as “as a minimum.”

Further embodiments can be found in the dependent claims.

Each output stage may comprise an IC circuit, preferably an IC control circuit. An IC circuit can be understood as an integrated switching circuit or an integrated circuit.

An IC can be connected upstream of each electronic switch. Preferably, the control unit is configured to control the IC switch(es). In other words, the output stages can be operated, activated or switched using the control unit. Alternatively, it is possible to use only one IC circuit for a set of output stages. The IC circuit or IC circuits can be included in the control unit.

A sine reference signal, also called an input signal, can be determined or calculated by the control unit and/or by means of a corresponding program. As an alternative to a digital determination, it is also possible to determine the sine reference signal in an analog manner.

In a preferred embodiment, the control unit is configured to record the step-shaped voltage curve. Preferably, the step-shaped voltage curve corresponds to an approximately sinusoidal voltage curve. In other words, the step-shaped voltage curve follows an approximately sinusoidal voltage curve. The more individual voltage curves, resulting in the step-shaped curve, that are generated, the more consistently a sinusoidal voltage curve can be generated. In other words, a sinusoidal voltage curve can be generated, which preferably has no, small or only a few jumps in its course.

An approximately sinusoidal voltage curve can be understood as a quasi, almost or nearly sinusoidal voltage curve, a voltage curve oriented towards the sinusoidal shape, or a sinusoidal voltage curve in principle.

In a further preferred embodiment, the target parameter is defined such that when a voltage from one of the electronic switch-generated voltage curves corresponds to the sine reference signal, switching occurs. This makes it possible to easily generate a plurality of pulse duration modulation output signals.

In a further preferred embodiment, each output stage has a bridge circuit or H-circuit connected upstream of the electronic switch. The bridge circuit can be configured as a half or full bridge. The half bridge can have two variable resistors, and the full bridge can have four variable resistors.

In a further preferred embodiment, the control unit for generating the step-shaped voltage curve from the sine reference signal has at least one processor. A processor is a programmable computing unit, or an electronic circuit that controls other machines or electrical circuits according to instructions given to it. Processors can be implemented as integrated circuits in the form of microprocessors and microcontrollers in embedded systems. As integrated circuits, CPLDs (complex programmable logic modules), ASICs (Application Specific Integrated Circuits) or FPGAs (Field Programmable Gate Array) can be used.

In a further preferred embodiment, the high-frequency signal generator is configured,
to generate sawtooth-shaped voltage curves according to the number of electronic switches,
to generate pulse duration modulation output signals corresponding to the number of sawtooth-shaped voltage curves, the target parameter being defined such that, when a voltage of a sawtooth-shaped voltage curve corresponds to the sine reference signal, switching takes place, and
to generate the step-shaped voltage curve from the pulse duration modulation square wave voltages based on the pulse duration modulation output signals. This makes it possible to easily generate pulse-width-modulated square wave voltages.

In a further preferred embodiment, the high-frequency signal generator is configured to smooth the step-shaped voltage curve using a low-pass filter in order to generate a sinusoidal voltage curve. By providing the low-pass filter, the step-shaped voltage curve can be easily smoothed. In this case, the control unit can be configured to actuate the low-pass filter.

In a further preferred embodiment, a low-pass filter, preferably consisting of a coil and a capacitor (LC element), is connected downstream of each electronic switch. In particular, the high-frequency signal generator is configured to smooth the step-shaped signal curve by means of the low-pass filter with the LC element, the quality of the sine wave being further increased.

In a further preferred embodiment, the plurality of output stages is at least 3 to 20, preferably 3 to 10, particularly preferably 3, 4, 6, 8 or 10. The more output stages used to generate the sawtooth-shaped voltage curves, the more finely the pulse duration modulation output signal can be graded until finally a continuous or nearly continuous sine wave is generated or mapped, or until the quality of the sine wave is optimal or nearly optimal.

Tests have shown that sufficiently good sine curves can be achieved with just 3 output stages. Tests have shown that the sine curves improve with 4, 6, 8 or 10 output stages. Tests have also shown that embodiments having more than 20 output stages have not shown any noticeable improvement in the shape of the sine curves in terms of smoothness. In principle, an optimization is carried out, it being possible for unevenness, disturbing waves, preferably disturbing harmonics, or jumps in the sine curve to be reduced. This can also be referred to as a sine approximation or a quasi-sine.

In a further preferred embodiment, the galvanic isolation is configured to smooth the signal curve output by the output stages. In other words, the transformer is configured to smooth the signal curve output by the output stages. The transformer can be used to smooth the signal in order to thereby eliminate possible bumps, jumps or outliers. For smoothing, a bandpass filter can be used, for example.

In a further preferred embodiment, the output stages are configured to be switched in succession. Switching can be understood as interconnection or energizing.

In a further preferred embodiment, the output stages are connected in parallel and/or in series.

In a further preferred embodiment, at least a first set of output stages having a plurality of first output stages, and a second set of output stages having a plurality of second output stages, are arranged in parallel with one another in the primary circuit.

This makes it possible to switch all first output stages first and then all second output stages. However, it is preferably possible to switch the first output stages and the second output stages alternately in succession.

The more output stages used to generate the sawtooth-shaped voltage curves, the more finely the pulse duration modulation output signal can be graded until finally a continuous or nearly continuous sine wave is generated or mapped, or until the quality of the sine wave is optimal or nearly optimal.

Preferably, the first set of output stages comprises at least two first output stages, and the second set of output stages comprises at least two second output stages. As mentioned above, tests have shown that with between 4 and 10 output stages, when the number of output stages is increased, the quality the sine curves could be improved. Tests have also shown that with more than 20 output stages there is no noticeable improvement in the shape of the sine curves in terms of smoothness.

Power can be supplied as follows:
energizing at least a first set of output stages with a plurality of output stages, and subsequently
energizing at least one second set of output stages with a plurality of second output stages, or
alternating energizing of one of the first and second output stages in each case, of both sets of output stages, in succession.

The object of the invention is further achieved by a system comprising an electrosurgical device as described above and an electrosurgical instrument, the electrosurgical instrument being configured for cutting and/or coagulating tissue.

Preferably, the electrosurgical instrument is a coagulation forceps. The coagulation forceps can be connected to the secondary circuit.

Using the system, it is possible to achieve rapid and reliable tissue sealing during minimally invasive endoscopy. A high frequency can be generated in the secondary circuit. This makes it possible to generate a sine wave with a frequency of at least 150 kHz, preferably at least 200 kHz, preferably at least 300 kHz.

The object of the invention is further achieved by a method for controlling an electrosurgical device for high-frequency generation with a frequency of at least 150 kHz, in a primary circuit of the device comprising a high-frequency signal generator with a plurality of output stages, each output stage comprising an electronic switch, and the primary circuit being galvanically isolated from a secondary circuit by means of a transformer, comprising the following steps:
switching the electronic switches depending on at least one target parameter,
by means of the high-frequency signal generator, generating a step-shaped voltage curve from pulse-width-modulated square wave voltages, which curve follows a sine reference signal, by means of pulse duration modulation from the totality of the voltage curves generated by the electronic switches, and
controlling an output voltage for actuating the output stages depending on the pulse-width-modulated square wave voltages by means of a control unit.

Preferably, the electronic switch is selected from the group comprising an electrical switch, an electrical circuit, a transistor or a MOS-FET.

In a preferred embodiment, the method comprises the following step
detecting the step-shaped voltage curve by means of the control unit, the step-shaped voltage curve corresponding to an approximately sinusoidal voltage curve.

In a further preferred embodiment, the method comprises the following step
switching when a voltage from one of the electronic-switch-generated voltage curves corresponds to the sine reference signal.

In a further preferred embodiment, the method comprises the following steps
generating sawtooth-shaped voltage curves corresponding to the number of electronic switches,
generating pulse duration modulation output signals corresponding to the number of sawtooth-shaped voltage curves, a target parameter being defined such that when the voltage of the sawtooth-shaped voltage curves corresponds to a sine reference signal, and
based on the pulse duration modulation output signals, generating the step-shaped curve from the pulse-width-modulated square wave voltages.

In a further preferred embodiment, the method comprises the following step
generating sawtooth-shaped signal waveforms, depending on the number of output stages, with preferably at least 3 to 20 sawtooth-shaped signal waveforms, preferably 3 to 10 sawtooth-shaped signal waveforms, and particularly preferably 3, 4, 6, 8 or 10 sawtooth-shaped signal waveforms.

In a further preferred embodiment, the method comprises next step
Smoothing the step-shaped voltage curve by means of a low-pass filter to generate a sinusoidal voltage curve, wherein preferably a low-pass filter, preferably consisting of a coil and a capacitor (LC element), is connected downstream of each electronic switch.

In a further preferred embodiment, the method comprises the following step
Smoothing the signal curve output by the output stages by means of galvanic isolation.

In a further preferred embodiment, the method comprises subsequent steps
energizing at least a first set of output stages with a plurality of output stages, and subsequently
energizing at least one second set of output stages with a plurality of second output stages, or
alternating energizing of one of the first and second output stages in each case, of both sets of output stages, in succession.

Energizing can be understood as switching.

The drawings, the description, and the claims contain numerous features in combination. It is understood that the features mentioned above and those to be explained below can be used not only in the combination specified in each case, but also in other combinations or on their own, without departing from the scope of the present invention. The invention is explained in more detail below with reference to embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of an electrosurgical device;

FIG. 2 is a schematic representation of a primary circuit from FIG. 1;

FIG. 3 is a pulse width modulation graph;

FIG. 4 is another pulse width modulation graph based on FIG. 2;

FIG. 5 is a schematic representation of an alternative primary circuit from FIG. 1;

FIG. 6 is a schematic representation of another alternative primary circuit from FIG. 1;

FIG. 7 is a schematic representation of yet another alternative primary circuit from FIG. 1,

FIG. 8 is a switching graph based on the arrangement of the output stages according to FIG. 7; and

FIG. 9 is a voltage graph based on FIG. 8.

### DESCRIPTION OF PREFERRED EMBODIMENTS

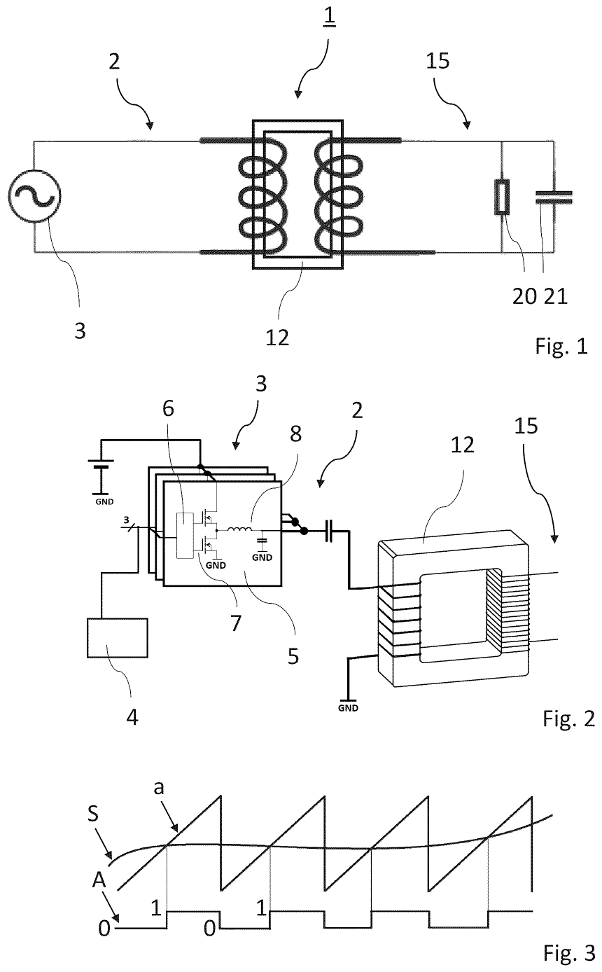

Referring to the drawings, FIG. 1 shows an electrosurgical device 1. The electrosurgical device 1 is used to generate high frequencies for coagulation and/or cutting of tissue. The frequency to be achieved with the electrosurgical device 1 is at least 150 Hz, preferably at least 200 kHz or preferably at least 300 kHz. The electrosurgical device 1 comprises a primary circuit 2 with a high-frequency signal generator 3, a secondary circuit 15, and a transformer 12. The primary circuit 2 is galvanically isolated from the secondary circuit 15 by means of the transformer 12. The secondary circuit 15 has a first ohmic resistor 20 and a second capacitive resistor 21. The resistors 20, 21 can be understood as tissue resistance. An instrument, e.g., coagulation forceps, (not shown) can be arranged or connected in the secondary circuit 15.

FIG. 2 is a schematic representation of the primary circuit 2 from FIG. 1 in detail.

The high-frequency signal generator 3, which is arranged in the primary circuit 2, comprises three output stages 5. Each of the three output stages 5 comprises an electronic switch 7, which is preferably configured as a MOS-FET, and an integrated circuit 6. The integrated circuits 6 are connected upstream of the electronic switches 7. The electronic switches 7 are controlled or activated by the integrated circuits 6. The electronic switches 7 are configured to be switched depending on at least one target parameter. A low-pass filter consisting of a coil and a capacitor (LC element 8) is connected downstream of each electronic switch 7. As further shown in FIG. 2, the high-frequency signal generator 3 comprises a control unit 4. The control unit 4 can be understood as a central control circuit. Preferably, the control unit 4 is configured to control or activate integrated circuits 6.

FIG. 3 is a pulse width modulation graph. A voltage curve a generated by an electronic switch 7 is formed as a sawtooth profile or it is sawtooth-shaped. The sawtooth profile can be understood as a counter that counts from 0 to a predetermined value, where it is then reset to 0. The increase in the voltage curve is linear, the decrease in the voltage curve is abrupt. After completing a cycle from ascent to descent, the cycle can then begin again.

A sine reference signal S (also input signal) can be determined or calculated in a processor of the control unit 4 and/or by means of a corresponding program. Alternatively, it is also possible to determine the sine reference signal S in an analog manner. Subsequently, a logical “and” connection is added. In this case, the sine reference signal S is linked to the sawtooth profile to generate a logical 1 starting at a certain counter reading. This generates a pulse duration modulation output signal in the form of a pulse-width-modulated square wave voltage A. However, a single pulse-width-modulated square wave voltage is not sufficient to generate a sinusoidal voltage curve or an approximately sinusoidal waveform sufficiently well.

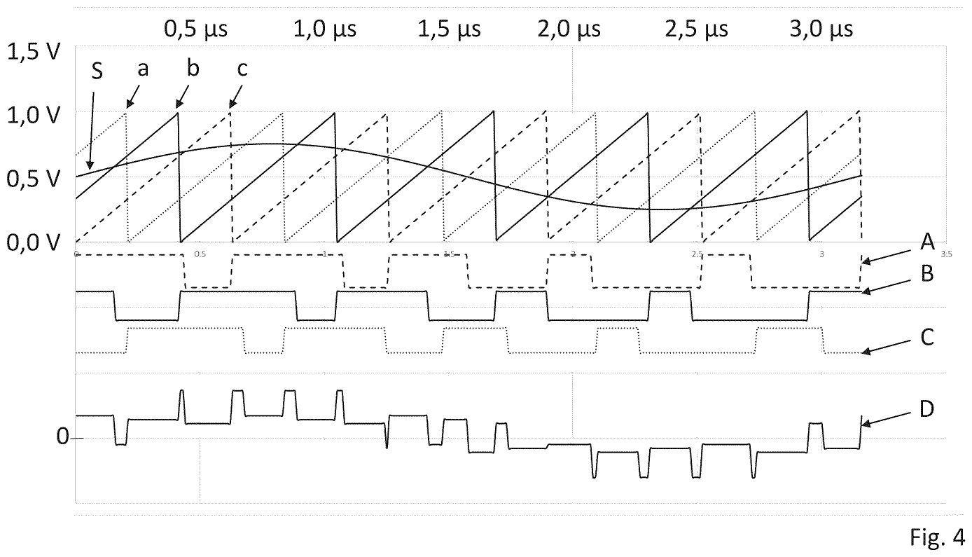

FIG. 4 is another pulse width modulation graph based on FIG. 2. Instead of just one pulse-width-modulated square wave voltage according to FIG. 3, three pulse-width-modulated square wave voltages A, B, C are now generated.

The high-frequency signal generator 3 is configured to generate a step-shaped voltage curve D from the pulse-width modulated square wave voltages A, B, C from the totality of the voltage curves a, b, c generated by the three electronic switches 7 by means of pulse duration modulation. In this case, the three electronic switches 7 are interconnected one after the other, i.e., in succession. The step-shaped voltage curve D follows the sine reference signal S. In order to generate the step-shaped voltage curve D from the sine reference signal S, the control unit 4 comprises at least one processor.

In detail, the high-frequency signal generator 3 is configured,
to generate the three sawtooth-shaped voltage curves a, b, c according to the three electronic switches 7,
to generate pulse duration modulation output signals corresponding to the three sawtooth-shaped voltage curves a, b, c, the target parameter being defined such that switching occurs when a voltage of a sawtooth-shaped voltage curve a, b, c corresponds to the sine reference signal S, and
starting from the pulse duration modulation output signals, to generate the step-shaped voltage curve D from the pulse-width-modulated square wave voltages A, B, C.

The control unit 4 is configured to regulate an output voltage for controlling the three output stages 5 depending on the pulse-width modulated square wave voltages A, B, C. Furthermore, the control unit 4 is configured to detect the step-shaped voltage curve D, this corresponding to or following an approximately sinusoidal voltage curve.

The above-mentioned target parameter is defined in such a way that switching occurs when a voltage generated by one of the electronic switches 7 corresponds to the sine reference signal S.

Each of the output stages 5 can have a half or full bridge connected upstream of the electronic switch 7. The transformer 12 can be configured to be operated by means of the half or full bridge in the primary circuit 2.

Furthermore, the high-frequency signal generator 3 is configured to smooth the step-shaped voltage curve D by means of a low-pass filter (LC element 8) in order to generate a sinusoidal voltage curve. In other words, the quality of the sine curve can be increased or improved using the low-pass filter.

The galvanic isolation is configured to further smooth the signal curve output by the output stages 5. In other words, the transformer is configured to further smooth the signal curve output by the output stages 5. This makes it possible to further smooth the voltage curve. In other words, the quality of the sine curve can be further increased or improved by means of galvanic isolation or the transformer.

The device 1 with at least the three output stages 5 is sufficient to generate a sinusoidal voltage curve or an approximately sinusoidal waveform sufficiently well.

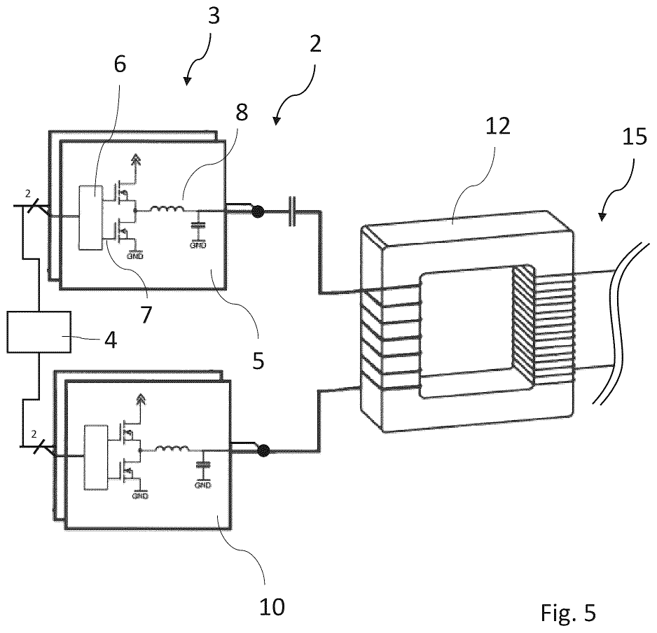

FIG. 5 is an alternative schematic representation of the primary circuit 2 from FIG. 1 in detail. In contrast to FIG. 2, instead of the three output stages 5, a total of four output stages 5, 10 are provided. In this case, a first set of output stages with two first output stages 5 and a second set of output stages with two second output stages 10 are arranged in parallel to one another in the primary circuit 2. The four electronic switches 7, which are preferably configured as MOS-FETs, are interconnected one after the other.

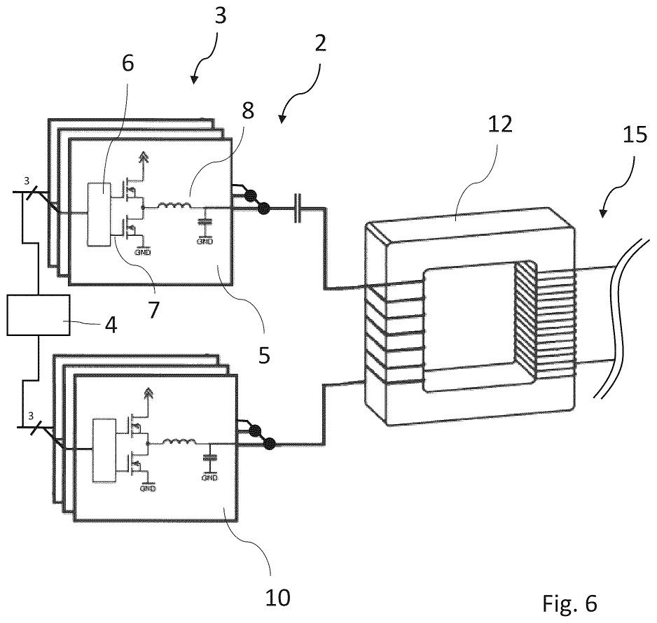

FIG. 6 is another alternative schematic representation of the primary circuit 2 from FIG. 1 in detail. In contrast to FIG. 2, instead of the three output stages 5, a total of six output stages 5, 10 are provided. In this case, a first set of output stages with three first output stages 5 and a second set of output stages with three second output stages 10 are arranged in parallel to one another in the primary circuit 2. The six electronic switches 7, which are preferably configured as MOS-FETs, are interconnected one after the other.

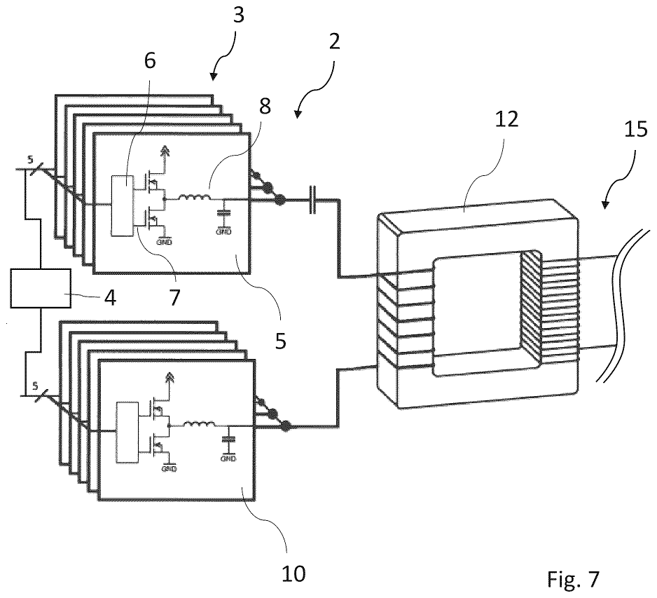

The following explanations regarding FIG. 7 also apply correspondingly to FIG. 5 and FIG. 6, which have fewer output stages than shown in FIG. 7.

FIG. 7 is another alternative schematic representation of the primary circuit 2 from FIG. 1 in detail. In contrast to FIG. 2, instead of the three output stages 5, a total of ten output stages 5, 10 are provided. In this case, a first set of output stages with five first output stages 5 and a second set of output stages with five second output stages 10 are arranged in parallel to one another in the primary circuit 2.

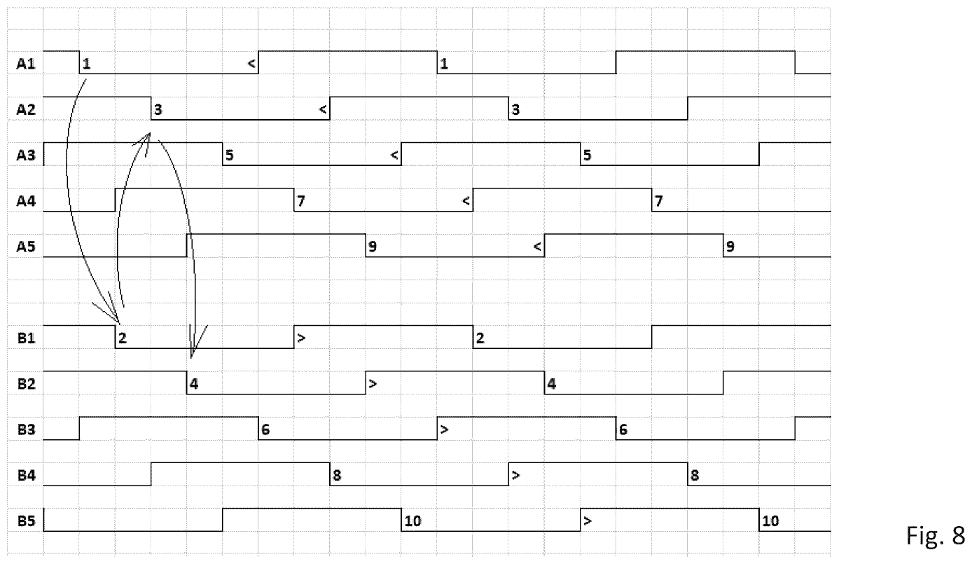

FIG. 8 is a switching graph over time based on the arrangement of the output stages 5, 10 according to FIG. 7. The first set of output stages with the five first output stages 5 is divided in the graph into the five first output stages A1 to A5. The second set of output stages with the five second output stages 10 is divided in the graph into the five second output stages B1 to B5. Switching or energizing takes place in succession according to the number sequence 1 to 10, as follows: A1, B1, A2, B2, A3, B3, A4, B4, A5 and B5. Once this cycle is completed, it starts again from the beginning.

The high-frequency signal generator 3 of FIG. 7 is configured, in accordance with the high-frequency signal generator 3 shown in FIG. 2, to generate a step-shaped voltage curve (not shown) from the pulse-width modulated square wave voltages A1, B1, to A5, B5 by means of pulse duration modulation from the totality of the voltage curves (not shown) generated by the ten electronic switches 7. The step-shaped voltage curve follows a sine reference signal S (not shown).

The control unit 4 is configured to detect the step-shaped voltage curve D, this corresponding to or following an approximately sinusoidal voltage curve.

In order to generate the step-shaped voltage curve D from the sine reference signal S, the control unit 4 comprises at least one processor, the control unit 4, as described above, being configured to regulate an output voltage for controlling the output stages 5 depending on the pulse-width-modulated square wave voltages A1, B1, to A5, B5.

The high-frequency signal generator 3 is configured to smooth the step-shaped voltage curve by means of a low-pass filter in order to generate a sinusoidal voltage curve.

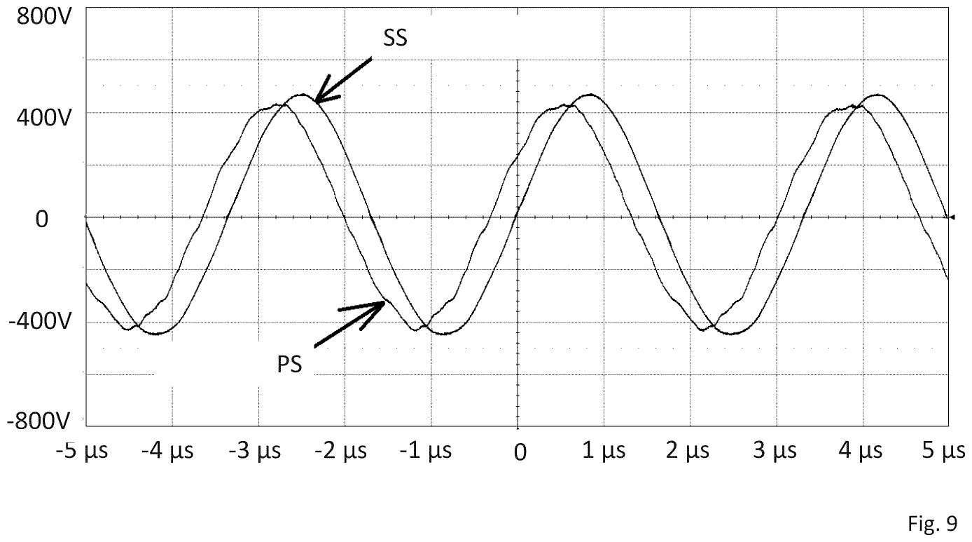

The voltage graph over time shown in FIG. 9 is based on the switching graph over time according to FIG. 8. The one sinusoidal curve SS (secondary voltage: 200 V/div) is much smoother than the other sinusoidal curve PS (primary voltage: 5 V/div), i.e., more continuous with few or preferably small jumps in the curve.

The invention makes it possible to generate a clean sine signal that has only low power losses in the electronics, generates no or only a few harmonics, and can be controlled quickly, safely, precisely, and reliably.

The invention relates to an electrosurgical device 1 for high-frequency generation, comprising a high-frequency signal generator 3 which has a plurality of output stages 5, each output stage 5 comprising an electronic switch 7, each of the electronic switches 7 being configured to be switched depending on at least one target parameter, the high-frequency signal generator 3 being configured to generate, by means of pulse duration modulation, from the totality of the voltage curves generated by the electronic switches 7, a step-shaped voltage curve comprising pulse-width-modulated square wave voltages which curve follows a sine reference signal, and a control unit 4 which is configured to regulate an output voltage for driving the output stages 5 depending on the pulse-width-modulated square wave voltages. The drawings, the description, and the claims contain numerous features in combination. It goes without saying that the above-mentioned features can be used not only in the combination specified in each case, but also in other combinations or on their own, without departing from the scope of the present invention.

While specific embodiments of the invention have been shown and described in detail to illustrate the application of the principles of the invention, it will be understood that the invention may be embodied otherwise without departing from such principles.

### REFERENCE CHARACTERS

1 device
2 primary circuit
3 high-frequency signal generator
4 control unit
5 output stage
6 IC (control circuit)
7 electronic switch
8 LC element
10 output stage
12 transformer
15 secondary circuit
20 resistor
21 resistor
a voltage curve
b voltage curve
c voltage curve
A square wave voltage
A1 square wave voltage
A2 square wave voltage
A3 square wave voltage
A4 square wave voltage
A5 square wave voltage
B square wave voltage
B1 square wave voltage
B2 square wave voltage
B3 square wave voltage
B4 square wave voltage
B5 square wave voltage
C square wave voltage
D step-shaped voltage curve
PS primary signal
S sine reference signal
SS secondary signal

1. An electrosurgical device for high-frequency generation with a frequency of at least 150 kHz for coagulation and/or cutting of tissue, electrosurgical device comprising:
a primary circuit with a high-frequency signal generator, wherein the high-frequency signal generator comprises a plurality of output stages;
a secondary circuit; and
a transformer, wherein the primary circuit is galvanically isolated from the secondary circuit by means of the transformer, wherein each output stage comprises an electronic switch, wherein each of the electronic switches is configured to be switched depending on at least one target parameter, wherein the high-frequency signal generator is configured to generate, by means of pulse duration modulation, from a totality of voltage curves generated by the electronic switches, a step-shaped voltage curve comprising pulse-width-modulated square wave voltages, which step-shaped voltage curve follows a sine reference signal; and
a control unit which is configured to regulate an output voltage for controlling the output stages depending on the pulse-width-modulated square wave voltages.

2. An electrosurgical device according to claim 1, wherein the control unit is configured to detect the step-shaped voltage curve, wherein the step-shaped voltage curve corresponds to an approximately sinusoidal voltage curve.

3. An electrosurgical device according to claim 1, wherein the target parameter is defined such that when a voltage generated by one of the electronic switches corresponds to the sine reference signal, switching takes place.

4. An electrosurgical device according to claim 1, wherein each output stage comprises a bridge circuit, connected upstream of the electronic switch.

5. An electrosurgical device according to claim 1, wherein the control unit comprises at least one processor for generating the step-shaped voltage curve from the sine reference signal.

6. An electrosurgical device according to claim 1, wherein the high-frequency signal generator is configured to
generate sawtooth-shaped voltage curves corresponding to a number of electronic switches
generate pulse duration modulation output signals corresponding to a number of sawtooth-shaped voltage curves, wherein the target parameter is defined such that, when a voltage of a sawtooth-shaped voltage curve corresponds to the sine reference signal, switching takes place, and
generate the step-shaped voltage curve from the pulse-width-modulated square wave voltages based on the pulse duration modulation output signals.

7. An electrosurgical device according to claim 1, wherein the high-frequency signal generator is configured to smooth the step-shaped voltage curve by means of a low-pass filter in order to generate a sinusoidal voltage curve.

8. An electrosurgical device according to claim 1, wherein a low-pass filter is connected downstream of each electronic switch.

9. An electrosurgical device according to claim 1, wherein the plurality of output stages is at least 3 to 20.

10. An electrosurgical device according to claim 1, wherein galvanic isolation is arranged to smooth a signal curve output by the output stages.

11. An electrosurgical device according to claim 1, wherein the output stages are configured to be switched in succession.

12. An electrosurgical device according to claim 1, wherein the output stages are connected in parallel and/or in series.

13. An electrosurgical device according to claim 1, wherein the output stages comprise at least a first set of output stages with a plurality of first output stages and a second set of output stages with a plurality of second output stages that are arranged in parallel to one another in the primary circuit.

14. A system comprising an electrosurgical device according to claim 1 and an electrosurgical instrument, wherein the electrosurgical instrument is configured for cutting and/or coagulation of tissue.

15. A method for controlling an electrosurgical device for high-frequency generation with a frequency of at least 150 kHz, wherein a primary circuit of the device has a high-frequency signal generator with a plurality of output stages, wherein each output stage comprises an electronic switch and wherein the primary circuit is galvanically isolated from a secondary circuit by means of a transformer, the method comprising the following steps:
switching the electronic switches depending on at least one target parameter,
generating, by means of the high-frequency signal generator, a step-shaped voltage curve from pulse-width-modulated square wave voltages, which curve follows a sine reference signal, by means of pulse duration modulation from the totality of the voltage curves generated by the electronic switches, and
regulating an output voltage for controlling the output stages depending on the pulse-width-modulated square wave voltages by means of a control unit.

16. A method according to claim 15, further comprising the following step-detecting the step-shaped voltage curve by means of the control unit, wherein the step-shaped voltage curve corresponds to an approximately sinusoidal voltage curve.

17. A method according to either claim 15, further comprising the following step—switching when a voltage from one of the electronic-switch-generated voltage curves corresponds to the sine reference signal.

18. A method according to claim 15 further comprising the following steps:
generating sawtooth-shaped voltage curves corresponding to the number of electronic switches,
generating pulse duration modulation output signals corresponding to the number of sawtooth-shaped voltage curves, wherein a target parameter is defined such that in each case the voltage of the sawtooth-shaped voltage curves corresponds to a sine reference signal, and
starting from the pulse duration modulation output signals, generating the step-shaped waveform from the pulse-width-modulated square wave voltages.

19. A method according to claim 15, further comprising the following step—generating sawtooth-shaped signal waveforms, depending on the number of output stages.

20. A method according to claim 15, further comprising the following step—smoothing the step-shaped voltage curve by means of a low-pass filter to generate a sinusoidal voltage curve.

21. A method according to claim 15, further comprising the following step-smoothing the signal curve output by the output stages by means of galvanic isolation.

22. A method according to claim 15, further comprising the following steps:
energizing at least one first set of output stages having a plurality of output stages, and subsequently; and
energizing at least one second set of output stages having a plurality of second output stages, or energizing one of the first and second output stages of both sets of output stages alternately in sequence.
